(19) United States
(12) Patent Application Publication
(10) Pub. No.: US 2005/0118698 A1
(43) Pub. Date: Jun. 2, 2005

(54) Multiplication of viruses in a cell culture

(75) Inventors: Jurgen Vorlop, Marburg (DE); Christian Frech, Marburg (DE); Holger Lubben, Wetter (DE); Jens-Peter Gregersen, Wetter (DE)

Correspondence Address: Chiron Corporation;Intellectual Property - R440, P.O. Box 8097, Emeryville, CA, 94662-8097, US

(73) Assignee: CHIRON BEHRING GMBH & CO., Marburg (DE)

(21) Appl. No.: 10/487,709

(22) PCT Filed: Sep. 11, 2002
(86) PCT No.: PCT/EP02/10207

(30) Foreign Application Priority Data
Sep. 12, 2001 (DE) 101 44 903.8

(51) Int. Cl. C12N 7/01; C12N 15/86

(52) U.S. Cl. 435/235.1; 435/456

(57) ABSTRACT

The present invention concerns methods for multiplication of viruses in cell culture in which cells are infected with a virus and after infection the cells are cultured in cell culture under conditions that permit multiplication of the viruses and at the same time targeted additional, at least two-fold, multiplication of the cells. The invention also concerns the use of the viruses so obtained or proteins expressed by them for production of drugs and diagnostic agents.

The invention concerns a method for multiplication of viruses in cell culture in which cells are infected with a virus and after infection the cells are cultured in a cell culture under conditions that permit multiplication of the viruses and at the same time targeted additional, at least two-fold, multiplication of the cells. The invention also concerns the use of the viruses so obtained or the proteins expressed by them for production of drugs and diagnostic agents.

Infectious diseases, especially viral infections, are still of major medical importance. The need to make available better methods by means of which viruses can be multiplied in culture in order to permit research on viruses and production of vaccines therefore remains unaltered. Production of vaccines in particular against viral infection ordinarily requires multiplication and isolation of large amounts of the corresponding virus.

Depending on the corresponding virus, different host systems and culture conditions for virus multiplication are used in the prior art. Standard host animals, embryonic chicken eggs, primary tissue cell cultures or established permanent cell lines are used as host systems (Rolle and Mayr (editors), Microbiology, Infection and Epidemic Science, 1978; Mahy (editor), Virology, A Practical Approach, 1985; Horzinek (editor), Compendium of General Virology, 1985).

Virus multiplication in embryonic chicken eggs is connected with high costs and time demands. The eggs must be incubated before infection and then tested for viability of the embryos. Only living embryos are capable of multiplying viruses. After infection with the virus being multiplied has occurred, and further incubation, the embryos are finally killed. The viruses isolated from the egg are freed of contaminants and concentrated. Since multiplication of viruses in incubated eggs is not possible under strictly sterile conditions, contaminating pathogenic microorganisms must be eliminated from the isolates if these are to be available for medical or diagnostic application.

An alternative to multiplication of viruses in chicken eggs is offered by eukaryotic host cells of defined cell lines (Gregersen, J. P., Pharmazeutische Biotechnologie, Kayser and Muller (editors), 2000, pp. 257-281). Numerous cell lines, however, are not suitable for production of vaccines or similar medically usable preparations owing to persistent foreign virus contaminations or because of the absence of demonstration of from viruses, unclear origin and history.

The methods used in the prior art for multiplication of viruses in cell culture all have the same basic scheme in which the cells are initially multiplied in the absence of the virus, then the virus is added and multiplied under conditions under which no significant multiplication of the cells occurs and the culture is harvested after maximum multiplication of the viruses.

For example, the Vero cells derived from kidney cells of monkeys were used for multiplication of individual viruses (polio virus, rabies virus) for vaccine production. These cells are available in different cell banks (like the American Type Culture Collection, ATCC) and are also made available by the World Health Organization (WHO) from a tested cell bank for medical research.

These Vero cells are adherent lines that require support surfaces for their growth, like glass bottles, plastic culture plates or plastic flasks. Growth on so-called microcarriers occurs in a culture of corresponding cells in the fermenter, i.e., generally small plastic spheres on whose surface the cells can growth.

It is known that adherent BHK (baby hamster kidney) and adherent MDCK (Mandine Darby canine kidney) cells and other cells can also actively multiply viruses, in addition to the aforementioned Vero cells, and are being used as substrate for production of pharmaceutical products, or their use is being considered. In the MDCK cell line ATCC CRL34 (NBL-2), in addition to influenza viruses, the vesicular stomatitis virus, the Coxsackie virus B5 (but not B3 or B4), reovirus [sic; retrovirus-typo in German] types 2 and 3, adenovirus types 4 and 5, as well as vaccinia viruses have also been experimentally multiplied. All corresponding publications, however, are geared exclusively toward adherent cultures (cf. ATCC product information). However, the suspension culture is preferred for multiplication of larger cell amounts, in which only the lymphoid and many transformed cells could thus far be multiplied in this system (Lindl (editor), Cell and Tissue Culture, 2000, pp.173ff). The MDCK cell line that is able to grow in suspension in protein-free culture media is disclosed in WO 97/37000. Multiplication of influenza viruses using the corresponding host cells is also described.

In addition to selection of an appropriate cell or host system, the culture conditions under which a virus strain is multiplied are also of great significance for the achievement of an acceptably high yield. To maximize the yield of desired virus strains, both the host system and the culture conditions must therefore be specifically adapted in order to achieve favorable environmental conditions for the desired virus strain. In order to achieve a high yield of different virus strains, a system that creates optimal growth conditions is therefore required. Many viruses are restricted to special host systems, some of which are very inefficient with respect to virus yield. Efficient production systems are often based on adaptations of the virus population of corresponding culture systems, often using intermediate stages with other host systems and employing protein additives—mostly serum of animal or human origin.

It is also known to experienced persons that nearly all cell cultures after initial multiplication with addition of serum or other growth factors can be kept at least for a certain time without serum or protein additives. For example, an arbitrary cell culture can be switched at the time of virus infection or right before harvesting to a medium without serum or protein additives and kept until harvest. This has been common practice for years in order to obtain virus material for vaccines or diagnostic tests while avoiding or reducing foreign proteins. Vaccines and cell cultures that were kept without this practice during the infection phase with addition of serum will have greater problems in being allowed for use in humans or animals, since the serum components can scarcely be adequately eliminated (cf. WHO recommendations “Proposed requirements for measles vaccine” (Live), Requirements for Biological Substances No. 12, revised 1978).

It is also known that many viruses can only be multiplied very poorly or not at all in protein-containing media. Viruses that rely on activity of proteolytic enzymes (proteases) for multiplication in culture systems are involved. Since these proteases are competitively inhibited by protein addition to the media, the addition of proteins at least from the time of infection or the production phase is logically out of the question here. Examples of viruses that must ordinarily be multiplied with addition of proteases and therefore to achieve good yields without protein additives to the infection medium, if possible, are influenza viruses and rotaviruses. Other types of viruses like paramyxoviruses and reoviruses can also benefit during multiplication from media that are as low in protein as possible (Ward et al. (1984), J. Clin. Microbiol. 748-753, “Efficiency of human rotavirus propagation in cell culture”). WO 96/15231 proposes cultivation of Vero and other cells in cell cultures in which a medium that gets by without the usual protein additives is to be used.

Other viruses are known to multiply poorly regardless of the medium composition and the culture conditions, for example rabies, rota-, pneumo-, or hepatitis A viruses (Provost and Hillemann, Proc. Soc. Exp. Bio. Med., 160:213-221 (1979); and Rolle and Mayr, loc. cit.).

After multiplication of the virus, it is usually isolated from the culture. Numerous methods are known in the prior art by means of which viruses, viral expression products or other proteins can be isolated after multiplication from the medium and/or the cells (Gregersen, loc. cit.; Mahy loc. cit.; Reimer, C. et al., Journal of Virology, December 1967, pp. 1207-1216; Navarro del Canizo, A. et al., Applied Biochemistry and Biotechnology, Vol. 61, 1996, 399; Prior, C. et al., BioPharm, October 1996, 22; Janson, Jan-C. and Ryden L. (editors), Protein Purification, 1997; and Deutscher, M. (editor), Methods in Enzymology, Vol. 182, 1990).

Based on the schematic course of the method (cell multiplication, virus multiplication, harvesting), the methods known in the prior art, however, only permit limited virus multiplication and harvesting.

The problem underlying the present invention therefore consists of providing methods for multiplication of viruses in cell culture that permit greater virus multiplication and simplified harvesting of larger amounts.

This problem has now been solved by the method for multiplication of viruses in which
(a) cells are infected with a virus;
(b) after infection the cells are cultured in cell culture under conditions that permit multiplication of the viruses and at the same time targeted additional, at least two-fold, multiplication of the cells.

Surprisingly, a significantly improved method for multiplication of viruses in cell culture is obtained if the course of the method is fundamentally changed, in that growth of the cells is also made possible during multiplication of the virus. This has the advantage that significantly more virus can be obtained in a shorter time. The output of the installation for cell culture is also improved.

According to the invention, cell culture is conducted so that the cells after infection are increased by at least two-fold or five-fold, preferably at least 10-fold.

Multiplication of the cells also means that culturing can be conducted over a period of at least 7 days after infection, but preferably the cells and viruses are multiplied over at least 21, 28 or 35 days in cell culture.

Depending on the method, it can be advantageous during multiplication of the viruses and the cells to add fresh medium, medium concentrate or media components at least once, or at least transfer part of the viruses and cells to a culture vessel that contains fresh medium, medium concentrate or medium components. However, it preferred to add fresh medium, medium concentrate or media components at least once during multiplication of the viruses and cells.

Addition of the medium, medium concentrate or media components is preferably repeated at least once or several times.

According to another preferred embodiment of the present invention, during culturing of the infected cells the culture medium is replaced with fresh culture medium or the culture volume is increased by adding fresh culture media. Exchange or replacement of the culture medium can also occur by medium concentrate or media components, like amino acids, vitamins, lipid fractions, phosphates and other substances. These steps can also be carried out repeatedly during culturing of the cells.

This permits an increase in virus yield by multiple virus harvest from the culture supernatant, and especially by increasing the total culture volume and also the cell count by adding fresh medium. Corresponding multiple harvests represent a significant advantage of the method according to the invention, since the yield of this system is significantly improved.

In the method of the present invention, MDCK cells are preferably used for multiplication of the virus.

In the cells used in the method according to the invention, cells that have the property of growing in suspension culture are involved. Cell lines that can also grow in the absence of support particles in the fermenter on a commercial scale are designated by this, which relative to other cells, have significant advantages during handling of the cultures, scale-up of the cultures and multiplication of viruses. Methods for adaptation of MDCK cells to suspension cultures are known in the prior art (WO 97/37000). The MDCK cells can originate from the cell line MDCK 33016.

According to another embodiment of the invention, cells that have the property both before and after infection of being adherent and growing as a suspension culture are used. This embodiment has the special advantage that a cell culture system and therefore a medium for development of cells from laboratory scale to commercial production can be used. Corresponding systems simplify drug registration significantly, since only the safety of an individual cell culture system need be checked.

The virus can have a genome from single-stranded deoxyribonucleic acid (ssDNA), double-stranded deoxyribonucleic acid (dsDNA), double-stranded ribonucleic acid (dsRNA) or single-stranded ribonucleic acid. The single-stranded ribonucleic acid molecules can then have the polarity of messenger RNA, RNA(+), or of opposite polarity, RNA(−).

The virus can be any virus known in the prior art. The viruses used in the context of the method according to the invention can be obtained from different collections like the ATCC (American Type Culture Collection) or the ECACC (European Collection of Animal Cell Cultures). Existing production strains or virus strains already premultiplied in cell culture are generally resorted to. Specific isolates can also be established but these are better suited for the corresponding application. According to one embodiment, the virus used in the method is chosen from the group consisting of: adenoviruses, ortho- and paramyxoviruses, reoviruses, picornaviruses, enteroviruses, flaviviruses, arenaviruses, herpes viruses and pox viruses. An adenovirus, polio virus, hepatitis A virus, Japanese encephalitis virus, Central European encelphalitis viruses, as well as the related eastern (Russian or other) forms, dengue virus, yellow fever virus, hepatitis C virus, rubella virus, mumps virus, measles virus, respiratory syncytial virus, vaccinia virus, influenza virus, rotavirus, rhabdovirus, pneumovirus, reovirus, herpes simplex virus 1 or 2, cytomegalovirus, varicella zoster virus, canine adenovirus, Epstein-Barr virus, as well as bovine or porcine herpes viruses, like BHV-1 or pseudorabies virus, can be used, in which the use of a rabies virus, rotavirus, pneumovirus or hepatitis A virus is particularly preferred.

According to another embodiment of the present invention, the genome of the virus can include a nucleic acid sequence that codes for a heterologous, functional protein with a size of at least 10 kd. Numerous vectors for expression of heterologous proteins are known in the prior art that are based on a viral genome, for example, on a herpes, vaccinia or adenovirus genome (Galler, R. et al., Braz. J. Med. Biol. Res., February 1997, 30(2):157-68; Willemse, M. J. et al., Vaccine, November 1996, 14(16):1511-6; Efstathiou, S., Minson, A. C., Br. Med. Bull., January 1995,.51(1):45-55; Hammerschmidt, W., Curr. Opin. Mol. Ther., October 2000, 2(5):532-9; Graham, Fl., Prevec, L., Mol. Biotechnol., June 1995, 3(3):207-20; Carroll, M. W., Moss, B., Curr. Opin. Biotechnol., October 1997, 8(5):573-7; Wojcik, J., Acta. Microbiol. Pol., 1995, 44(2):191-6; Ramirez, J. C. et al., J. Virol., August 2000, 74(16):7651-5; Hagen, Anna et al., Biotechnol. Prog., 1996, 12, 406-408; Huyghe, Bernard et al., Human Gene Therapy, November 1995, 6:1403-1416).

In the context of the present invention, methods for multiplication of those viruses in which the viral genome was altered by addition or substitution of sequences so that the genome codes for a heterologous functional protein with a size of at least 10 kd, i.e., not originally belonging to the virus, are also included. According to the invention, a protein is referred to as a functional protein when the protein is at least capable of triggering an immune reaction against this protein. Naturally the protein can have additional biological activities in addition to immunological activity, for example, act as an enzyme or cytokine.

The viruses used in the method according to the invention can also have deletions of individual genes in the viral genome. For example, genes of a virus to be used as a vaccine that code for pathogenicity factors can be deliberately deleted. Corresponding deletions preferably include no more than 500 or 1000 nucleotides.

Naturally the virus employed by the method according to the invention can also include a complete viral genome.

Multiplication of the viruses in suspension culture can occur according to the method of the invention in the presence or absence of serum in the medium. Special advantages are obtained by the absence of serum, since these cell culture conditions significantly simplify registration of medical use of the product so produced. By dispensing with serum additions to the culture medium, costly purification steps to eliminate medium contaminations are also avoided. Improvements with respect to quality of the product are therefore also achieved and costs are avoided on this account.

A medium is referred to as a serum-free medium in the context of the present invention in which there are no additives from serum of human or animal origin.

Specific proteins that do not have an interfering effect on the culture and subsequent use can be added in defined amounts to corresponding cultures. This type of culture medium is referred to as a chemically defined medium. Selected proteins, like mitogenic peptides, insulin, transferrin or lipoproteins are added to this medium, which can be obtained from different producers known to one skilled in the art. Mitogenic peptides in the context of the present invention are preferably understood to mean plant hydrolyzates, for example, soybean protein hydrolyzate or lysates from proteins of other useful plants.

According to a particularly preferred embodiment, however, the media are fully protein-free. Protein-free is understood to mean cultures in which multiplication of the cells occurs with exclusion of proteins, growth factors, other protein additives and non-serum proteins. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

According to a preferred embodiment of the present invention, during culturing of the infected MDCK cells, fresh medium, medium concentrate or media ingredients like amino acids, vitamins, lipid fractions or phosphates are added.

The method according to the invention can then be conducted in a perfusion or batch system. Culture systems in which the medium is continuously supplied and withdrawn are referred to as perfusion systems. As an alternative to this, the cells can also be cultured in a batch system in which the system is run as a largely closed system without supplying medium from inoculation to harvesting.

The cell culture conditions to be used for the desired application (temperature, cell density, pH value, etc.) are variable over a very wide range owing to the suitability of the cell line employed according to the invention and can be adapted to the requirements of the application. The following information therefore merely represents guidelines.

Multiplication of the cells before infection can be conducted starting from seed cultures or small culture vessels in a perfusion system using ordinary support methods like centrifugation or filtration. It has proven advantageous to exchange the culture medium during primary culture of the cells in such a system with a rate of up to three fermenter fillings per day. The cells can be multiplied under these conditions up to cell densities of 2×10⁷. Control of the perfusion rate occurs during culturing preferably by means of parameters known to one skilled in the art, like cell count, glutamine, glucose or lactate content.

When a batch system is used, cell densities up to about 8-25×10⁵ cells/mL can be reached at a temperature of 37° C. and a generation time of 20 to 30 h.

Moreover, the cells can be multiplied according to the invention in a fed-batch system before infection. In the context of the present invention, a culture system is referred to as a fed-batch system in which the cells are initially cultured in a batch system and depletion of nutrients (or part of the nutrients) in the medium is compensated by controlled feeding of concentrated nutrients. In a fed-batch system the cells can be multiplied to a cell density of about 1-10×10⁶.

It has also proven advantageous to adjust the pH value of the medium during multiplication of cells before infection to a value between pH 6.6 and pH 7.8 and especially between a value between pH 7.2 and pH 7.3.

Culturing of cells before infection preferably occurs at a temperature between 30 and 40° C. and especially at a temperature between 33 and 37° C. The oxygen partial pressure is adjusted during culturing before infection preferably at a value between 25 and 95% and especially at a value between 35 and 60%. The values for the oxygen partial pressure stated in the context of the invention are based on saturation of air.

It has proven advantageous for the method according to the invention that infection of cells occurs at a cell density of preferably about 8-25×10⁵ cells/mL in the batch system or preferably about 5-20×10⁶ cells/mL in the perfusion system. The cells can be infected with a viral dose (MOI value, “multiplicity of infection”; corresponds to the number of virus units per cell at the time of infection) between 10⁻⁸ and 10, preferably between 0.0001 and 0.5.

Culturing of the cells after infection can also occur in the perfusion, batch or fed-batch system. The same culture conditions as used before can be used (temperature between 30 and 40° C., oxygen partial pressure between 5 and 100%, pH value of the medium between pH 6.6 and pH 7.8).

Methods are also made available according to the invention that include harvesting and isolation of viruses or the proteins generated by them. During isolation of viruses or proteins, the cells are separated from the culture medium by standard methods like separation, filtration or ultrafiltration. The viruses or the proteins are then concentrated according to methods sufficiently known to those skilled in the art, like gradient centrifugation, filtration, precipitation, chromatography, etc., and then purified. It is also preferred according to the invention that the viruses are inactivated during or after purification. Virus inactivation can occur, for example, by β-propiolactone or formaldehyde at any point within the purification process.

The method according to the invention is especially suited for production of drugs, especially for production of vaccines and diagnostic agents.

Production of the drug can include multiplication and isolation of the virus or protein produced by it and mixing with an appropriate adjuvant, auxiliary, buffer, diluent and/or drug carrier. Adjuvants in the context of the present invention are understood to mean substances that increase immune response. These include hydroxides of various metals, like aluminum hydroxide, components of the bacterial cell wall, oils or saponins. The vaccines are particularly suited for prophylactic or therapeutic treatments of viral infections.

The immunogenicity and/or efficacy of the corresponding vaccines can be determined by methods known to one skilled in the art, like protective experiments with loading infection or determination of the antibody titer necessary for neutralization. Determination of the virus amount or amount of antibodies produced can occur by determination of the titer or amount of antigen according to standard methods sufficiently known to one skilled in the art, like virus titration, hemagglutination test, antigen determination or protein determination of different types.

The methods according to the invention are also suitable for production of a diagnostic composition. The compositions can include a virus obtained from the method or a protein produced by it. In combination with additives common in the prior art and detection reagents, these compositions can be used as a diagnostic test that is suitable for virus or antivirus antibody detection.

All the virus titers in the following examples were determined according to the final dilution method and statistical 50% end point determination according to Spearman-Kaerber, known to one skilled in the art (cf. Horzinek, Compendium of General Virology, 2^{nd} edition, 1985, Parey Verlag, pp. 22-23). Eight test cultures were infected in microtiter plates with 100 μL amounts of a virus dilution, in which dilutions of the virus material from 10⁻¹ to 10⁻⁸ were used. Evaluation of the virus titrations occurred either microscopically by means of the cytopathic effect as test cultures or with immunological detection methods employing virus-specific antibodies. Binding of the virus-specific antibodies is made visible as immunofluorescence with fluorescein-labeled antibodies or using biotin-labeled secondary antibodies and a streptavidin/biotin/peroxidase amplifier complex, as well as a precipitatable dye (Gregersen et al., Med. Microbiol. Immunol., 177:91-100). The unit of virus titer is the culture-infectious dose 50% (CID₅₀). The virus-specific detection cells used for the different types of virus and, if applicable, the immunological detection methods are mentioned in the virus-specific examples.

### EXAMPLES

### Example 1

### Handling of the Cell Culture System as a Suspension Culture in the Early Working Steps and on a Laboratory Scale

MDCK cells from seed cell vials stored in liquid nitrogen were quickly thawed by immersion in a water bath and immediately diluted in culture medium (Ultra CHO with supplement, BioWhittaker, standard medium) with a cell count of about 1×10⁵ cells/mL, generally about 1:100. The cells were then separated from the medium, taken up in fresh medium by centrifugation (10 min at 800 G) again and poured into spinner culture bottles (100 mL working volume, Bellco or Techne). These culture lots were incubated at 37° C. on a magnetic stirrer at 50-60 rpm. Cell growth was monitored by checking the cell count. On reaching cell counts of 8×10⁵ for a maximum of 1.6×10⁶ cells/mL, the cultures were transferred by dilution of the cells in fresh standard medium and seeding new spinner culture bottles of 100 to 1000 mL working volume and incubated until the maximum or desired cell densities were reached during agitation as described above. In these cell passages, the dilution of the corresponding culture was adapted to the type of cell growth in the range between 1:4 and 1:10 so that the maximum cell count was reached, as required, within 3 to 5 days. As an alternative, this type of cell culture was tried without addition of supplements to the medium and could be maintained without problems over at least 10 passages.

### Example 2

### Handling of the Cell Culture System as an Adherent Culture

Established suspension cultures (cf. Example 1) were diluted in different media so that the cell count was about 1×10⁵ cells/mL and then poured into a variety of cell culture vessels (see Table 1). The cell culture volumes then corresponded to the usual amounts with a corresponding culture vessel, i.e., about 4 mm culture medium over the seeding surface or about 1 mL of medium for 2.5 cm² of culture surface. The cultures were generally incubated at the temperature of 37° C. common for most cell cultures, but significant deviations of incubation temperature were also possible without noticeable loss (see Table 1). The culture systems tested, as well as the results in cell growth achieved with them are shown in Table 1 and indicate that the cell system behaves roughly the same and robustly in various media and culture systems.

Monolayer cultures produced in this way were used for titration of virus harvests in microtiter plates and for culturing of viruses under microscopic control or for immunofluorescence investigation, hemadsorption tests and other virological or immunological standard methods that can be conducted better in adherent one-layer cultures than in suspension cultures. In addition, such cultures were particularly suitable for recovering pure virus strains by plaque purification or diluting out. Finally, the adherent cultures were also used for virus multiplication on small and large scales; larger amounts preferably in roller bottles.

TABLE 1
Cell growth in various adherent culture systems.

| | | | | | Confluent culture |
|---|---|---|---|---|---|
| Cell culture | Cell seeding | Media | Additives | | after days |
| system | (×10⁵ cells/mL) | employed | employed | Incubation^{#} | (8-20 × 10⁵ cells/mL) |
| Plastic culture | 0.8-1.0 | MEM, EDM, | 1-5% FCS or | 33 or 37° C. | 4-5 |
| flasks | | Opti-MEM*, | Supp.* | | |
| | | Ultra CHO* | | | |
| Plastic culture | 2.0 | MEM, EDM, | 1-5% FCS or | 33 or 37° C. | 2-3 |
| flasks | | Opti-MEM*, | Supp.* | | |
| | | Ultra CHO* | | | |
| Microtiter | 2.0-4.0 | MEM, EDM, | 0.5-3% FCS or | 33 or 37° C. | 1-2 |
| plates | | Opti-MEM*, | Suppl.* | | |
| | | Ultra CHO* | | | |
| Microtiter | 2.0-4.0 | MEM, EDM, | 1% FCS for | 37° C. | 1 |
| plates | | Opti-MEM*, | 1 day, then | | |
| | | Ultra CHO* | without | | |
| | | | additives | | |
| Roller bottles | 1.0 | EDM, Opti- | 0.5-3% FCS or | 33 or 37° C. | 4-5 |
| | | MEM*, Ultra | Supp.* | | |
| | | CHO* | | | |
| Roller bottles | 1.0 | EDM, Opti- | 1% FCS or | 33 or 37° C. | 5-7 |
| | | MEM*, Ultra | Supp.* for | | |
| | | CHO* | 3 days, then | | |
| | | | without | | |
| | | | additives | | |
| Spinner + microcarrier | 2.0 | BME | 0.5-3% FCS or | 33 or 37° C. | 5-7 |
| | | MEM | Supp.* | | |
| | | EDM | | | |

BME: Basal Medium Eagle; bicarbonate supplement (2-2.5% of a 5% stock solution)
MEM: Minimum Essential Medium; bicarbonate supplement (2-2.5% of a 5% stock solution)
EDM: Dulbecco's Modified Eagle Medium; bicarbonate supplement (2-2.5% of a 5% stock solution)
FCS: fetal calf serum
Supp.: Ultra CHO supplement
^{#}adjusted value; the actually measured values with deviations by +2 and −3° C.
*manufacturer: Bio-Whittaker

### Example 3

### Virus Isolation, Recovery and Production of Seed Virus Preparations

Primary isolates, like virus-containing organ, tissue or tissue fluid samples, throat swabs or stool samples were suspended in an ice bath in standard medium (any other media or phosphate buffers are likewise possible) with addition of antibiotic (PSN: 100 U/mL penicillin, 100 μg/mL streptomycin, 50 μg/mL neomycin) and homogenized, if necessary (finely ground with mortars, scalpel blades or a so-called Douncer or Potter homogenizer). The suspension obtained was filtered with an ordinary laboratory syringe filter adapter with a pore size of 0.45 μm (for isolation of smaller, uncoated viruses also 0.2 μm). The filtrate was inoculated in small culture flasks (25 cm², see Example 2) with fresh culture medium. To increase the yield several cultures were provided with an inoculum of 100 μL to 1 mL and then incubated at 37° C. For virus isolates from the upper respiratory tract, it is recommended to prepare additional cultures at a lower incubation temperature of 33° C.

Pure virus isolates already multiplied in the culture were used for infection directly in the culture system according to the invention according to Examples 1 or 2. However, since a higher virus content of the virus preparation could be assumed here, smaller inoculum amounts of 100 μL or less were generally used. A MOI (multiplicity of infection) of 0.1 and 0.01 was preferred for such first infections in the culture system according to the invention; infection with MOI in steps diminishing by a factor of 10 from 10 to 0.0001 was repeated when the result was unsatisfactory.

The infected cultures were then examined daily with a microscope for virus-related cell damage (CPE, cytopathic effect) and compared with control cultures. As an alternative in viruses that cause no CPE, the culture was examined for the presence of specific virus antigens or their genes (e.g., specific HA tests depending on the type of virus; ELISA, PCR). After three to four days or a positive finding (shrinkage of the cells, cell death, rounding and dissolution of the cell lawn in adherent cultures, plaque formation), cell-free centrifuged culture supernatants were frozen as samples, and with a negative or doubtful finding on the other hand the entire culture was adjusted with fresh medium to a cell count of 1×10⁵ cells (dilution of suspension cultures or trypsin treatment of the adherent cultures with subsequent dilution of the individual cells) and further incubated distributed in new cultures. Since this corresponded in most media to a dilution of the cultures of 1:4 to 1:20, to avoid logarithmic multiplication of the number of cultures, after the second such culture passage at the latest only a part of the possible cultures were further maintained. After three to four passages, virus isolates could be successfully isolated and detected from the appropriate virus-containing starting material.

For most virus types, depending on the virus content and quality of the starting material, a virus-related CPE was found after 2 to 7 days of incubation (see also virus-specific examples). Some viruses, however, multiply very slowly or exhibit no CPE and must therefore be detected by extended passages and incubation times or a specific test (the required methods are listed under the specific virus examples). As an example for a virus without CPE with slow multiplication which also requires a special detection system, the special example of hepatitis A virus is referred to. The detection test described there is also suitable for detection of other viruses, especially those without specific CPE, when corresponding antisera are used.

Practically, a newly isolated virus should only be used after three-fold plaque purification or preparation of a pure isolate by the so-called limited dilution technique. The methods required for this can be taken from specialist textbooks according to the prior art (see e.g., B. W. Mahy: Virology—A practical approach; IRL Press, Oxford, 1985).

If appropriate virus preparations are available from the primary isolate or as an established strain, these are then used for infection of spinner cultures in order to recover a homogenous seed virus for production purposes. Without restricting ourselves to the object of the invention, a first infection is initially recommended in small spinner cultures with 100 mL culture medium with MOIs from 10 to 0.00001, preferably 0.1 to 0.0001. The most favorable conditions (especially with reference to MOIs and harvest times) to achieve more rapid and higher virus values or yields were chosen in order to produce a seed virus in a culture system of the required size in an additional virus passage according to the prescribed production scale and number of production runs. Depending on the virus yields achieved and the production time prescribed, the scale for this seed virus passage could be from a few spinner cultures to a 1000 mL scale to small fermenters up to roughly 10 L of volume or more. The harvested virus was freed of any cell residues by filtration or centrifugation and aliquoted into small amounts suitable for production and stored at temperatures below −70° C., if possible.

### Example 4

### Handling of the System as Adherent Microcarrier Culture for Production Purposes

Culturing of adherent MDCK cells occurred in roller bottles according to Example 2, Table 1 with BME plus 3% fetal calf serum (FCS). After culturing in the system, the cells were separated from the surface of roller bottles. This occurred enzymatically with an appropriate trypsin solution with ordinary methods known to one skilled in the art. As an alternative, according to Example 1, suspension cells were cultured in the spinner cultures and used directly to coat the microcarrier.

The production fermenter was filled with microcarriers of the Cytodex 3 type (Pharmacia). The microcarrier (specific weight 5 g/L) was autoclaved and conditioned with nutrient media. The method guaranteed adhesion of the cells to the surface of the microcarrier. The cells recovered in this manner were transferred to the production system so that the cell density was 1×10⁵ cells/mL. The cells adhered to the microcarrier and were cultured to confluence or to achieve a cell density of 3×10⁶ cells/mL.

After the cell culture phase, the nutrient medium present was replaced with fresh nutrient medium. For this purpose, protein-free nutrient media were used. Two wash cycles were run. A wash cycle consisted of turning off the agitator, settling of the microcarrier, removal of the nutrient medium consumed, addition of fresh nutrient medium and resuspension of the microcarrier. After the washing step the cell culture was mixed with trypsin (2.5 mg/L).

Infection of the cell culture with seed virus then occurred. This seed virus was obtained and used according to Example 3. The MOI was then virus-specific and amounted to between 0.1 and 0.000001, preferably between 0.01 and 0.001. After the end of the infection phase, whose time, on the one hand, is determined by the specific virus (see specific examples) and, on the other hand, also by the MOI chosen, the agitator was stopped and the microcarriers sedimented. The virus-containing supernatant was taken off and purified by appropriate separation methods from cell residues. For cell separations, ordinary centrifuges or separators, filters and crossfiow filtration units known to one skilled in the art were used.

### Example 5

### Handling of the System as Suspension Culture up to a Production Volume on a 1000 L Scale Using Serum-free Medium

Culturing of suspension cultures for a production volume of 1000 L occurred with spinner bottles (Techne Co.) on a small scale to 1000 mL culture volume (see Example 1). The cell density in the spinner was 1×10⁵ cells/mL. The cells were cultured in the batch process and transfer at a cell density of 1×10⁶ cells/mL by simple dilution in fresh medium in a 1:10 ratio. Serum free medium (Ultra CHO, BioWhittaker) was used as medium for cell culture. From a volume of 10 L agitated fermenters (30 agitator revolutions per minute) with permanent alration and temperature control (control temperature 37° C. for a cell culture), pH value (control range 7.1 to 7.3) and oxygen partial pressure (45 to 55% pO₂) were used (technical details as in Table 2). The scale-up volumes were 10 L, 100 L, 1000 L according to the transfer ratio of 1:10. The fermenters reached the final cell density of 1×10⁶ cells/mL and a time of 3 to 4 days at an initial cell density 1×10⁵ cells/mL. On a 1000 L scale, a fed-batch was additionally conducted with glucose solution (100-200 g/L) in order to increase the cell density to 3×10⁶ cells/mL. The cell yields achieved are shown in comparison in Table 2.

### Example 6

### Handling of the System as Suspension Culture to Production Volumes up to a Volume of 1000 L Using Chemically Defined Medium

Culturing of the suspension cultures for a production volume of 1000 L occurred as described in Example 5. On the other hand, a chemically defined medium (ProCHO4CDM) was used as an alternative for cell culture. It proved to be advantageous to conduct three to five prepassages for adaptation in this medium. The cell yields achieved are compared in Table 2.

### Example 7

### Handling of the System as a Suspension Culture up to a Production Volume on a 1000 L Scale Using a Protein-free Medium

Culturing of the suspension cultures for a production volume of 1000 L occurred as described in Example 5. Protein-free medium (SMIF7, Life Technologies) was used as medium for cell culture. It proved to be advantageous to run 5-10 prepassages for adaptation in this medium. The cell yields achieved are compared in Table 2.

TABLE 2
Culturing of cells (MDCK 33016) for a production scale in a fermenter
using various methods and media.

| No. | Method | Medium | N/T/pO₂/pH | X₀ | X |
|---|---|---|---|---|---|
| 1 | Batch | Ultra CHO | 30 min⁻¹ | 1 × 10⁵ mL⁻¹ | 1 × 10⁶ mL⁻¹ |
| | | | 37° C. | | |
| | | | 45-55% | | |
| | | | 7.1-7.3 | | |
| 2 | Fed-batch | Ultra CHO | 30 min⁻¹ | 1 × 10⁵ mL⁻¹ | 3.1 × 10⁶ mL⁻¹ |
| | | | 37° C. | | |
| | | | 45-55% | | |
| | | | 7.1-7.3 | | |
| 3 | Batch | ProCHO4CDM | 30 min⁻¹ | 1 × 10⁵ mL⁻¹ | 1 × 10⁶ mL⁻¹ |
| | | | 37° C. | | |
| | | | 45-55% | | |
| | | | 7.1-7.3 | | |
| 4 | Fed-batch | ProCHO4CDM | 30 min⁻¹ | 1 × 10⁵ mL⁻¹ | 3.3 × 10⁶ mL⁻¹ |
| | | | 37° C. | | |
| | | | 45-55% | | |
| | | | 7.1-7.3 | | |
| 5 | Batch | SMIF7 | 30 min⁻¹ | 1 × 10⁵ mL⁻¹ | 1 × 10⁶ mL⁻¹ |
| | | | 37° C. | | |
| | | | 45-55% | | |
| | | | 7.1-7.3 | | |
| 6 | Fed-batch | SMIF7 | 30 min⁻¹ | 1 × 10⁵ mL⁻¹ | 3.0 × 10⁶ mL⁻¹ |
| | | | 37° C. | | |
| | | | 45-55% | | |
| | | | 7.1-7.3 | | |

X₀: Initial cell density
X: Final cell density
N/T/pO₂/pH: Agitator speed, temperature, oxygen partial pressure, pH value

### Example 8

### Handling of the System in the Production Phase with Serum-free Medium

After culturing of suspension cultures to a production scale according to Example 5, the cells were distributed to three fermenters of equal volume 3×1000 L and filled with fresh medium. Each fermenter received ⅓ volume of preculture and ⅔ volume of fresh medium. The same medium as in the culturing phase was used (UltraCHO, BioWhittaker). After filling, the cell culture was mixed with 10 mg/L trypsin. Infection of the cell culture with a seed virus (influenza B/Harbin/7/94) then occurred at a MOI of 0.001 and further incubation under the same fermentation conditions as during cell culture, but at 33° C., over 96 h. The cell-containing supernatant was then taken off and the cells then separated with a separator. An additional filtration step occurred through a cartridge filter with a pore size of 0.45 μm to separate additional fine particles.

The virus harvests were tested for virus content with standard methods in the HA test with 0.5% chicken erythrocytes and by virus titration in adherent MDCK cells: the measured HA content was 1024 U, the virus titer was 108.2 CID₅₀/mL.

### Example 9

### Handling of the System in the Production Phase with Chemically-defined Media

Preparation of the production cells occurred as described in Example 8. However, chemically defined medium (ProCHO4CDM, BioWhittaker) was used as fresh medium. After filling, the cell culture was mixed with 2.5 mg/L trypsin. Subsequent infection was conducted as described in Example 8.

The measured HA content was 1024 U, the virus titer was 107.5 CID₅₀/mL.

### Example 10

### Handling of the System in the Production Phase with Protein-free Medium

Preparation of the production cells occurred as described in Example 8. However, protein-free medium (SMIF7, Life Technologies) was used as fresh medium. After filling, the cell culture was mixed with 2.5 mg/L trypsin.

Subsequent infection was conducted as described in Example 8. The measured HA content was 1024 U, the virus was titer 10^{7.9} CID₅₀/mL.

### Example 11

### Culturing and Infection with Chemically-defined Media

Culturing of the cells occurred as described in Example 6, infection as described in Example 9. The total cell culture from culturing to harvesting of the infection therefore occurred in chemically-defined medium.

### Example 12

### Culturing with Chemically-defined Media and Infection in Protein-free Medium

Culturing of the cells occurred as described in Example 6 in chemically-defined medium, infection as described in Example 10 in protein-free medium.

### Example 13

### Culturing and Infection in Protein-free Medium

Culturing of the cells occurred as described in Example 7, infection as described in Example 10. The entire cell culture from culturing to harvesting of the infection occurred in protein-free medium.

### Example 14

### General Description of Virus Purification

After conclusion of the virus multiplication phase, the cell culture harvest was filtered through a deep bed filter with a pore size of 0.45 or 0.5 μm in order to separate cells and cell fragments. As an alternative this separation was conducted with a separator. The viruses contained in the clarified harvest were concentrated and purified if necessary by ultrafiltration, in which a membrane with an exclusion limit between 50,000 and 1,000,000, preferably 100,000 to 500,000, was used. The virus concentrate obtained was loaded on a chromatography column packed with CS (Cellufine Sulfate, Millipore). After contaminants were eliminated by washing with buffer, the viruses were eluted with a 0.3 to 3M NaCl solution. The eluate was desalted by ultrafiltration and further concentrated. As an alternative or in combination with chromatographic purification, an additional purification effect can be achieved by ultracentrifagation. Most viruses can also be purified according to their buoyant density by ultracentrifugation in a sucrose gradient with subsequent fractionation of the gradient. Virus inactivation with formaldehyde or β-propiolactone can be introduced at any point within the purification process, but preferably is used after concentration or after purification, since the volumes being inactivated are then already substantially reduced.

### Example 15

### Recovery of Inactivated Pure Virus Preparation for Formulation of Vaccines

Flaviviruses (Central European encelphalitis virus, strain K 23) were cultured according to Examples 5, 6 and 7 in different media at an inoculation dose of 0.2 MOI (for details, cf. Example 22).

The harvested, virus-containing culture medium was freed of any cell residues present by centrifugation and filtration via filters with a pore size of 0.45 μm. For safety reasons, this material was already inactivated after filtration by addition of β-propiolactone in a dilution of 1:2000 or 1:2500 and incubation at 2-8° C. for 24 h. A cell culture test of the inactivated preparations after 2 h of hydrolysis of the inactivation agent at 37° C. showed that no active virus was present up to a detection limit of less than 0.03 infectious units/mL.

For analysis of the subsequently described purification steps, a BCA [bicinchoninic acid] assay (Pierce) was used to determine the total protein content. The specific antigen content was determined with a sandwich ELISA using specific monoclonal antibodies against the E-glycoprotein (Niedrig et al., 1994, Acta Virologica 38:141-149) and a polyclonal antiserum in-house produced against purified virus from rabbits. The values for the inactivated starting material were then used as reference value (corresponding to 100%).

Purification by gradient centrifugation:

Inactivated virus preparations were purified according to known methods by density gradient ultracentrifugation (15-60% sucrose) at 80,000 G. The gradient was then fractionated and in samples of the fractions the extinction at 280 nm was determined to identify the virus peak. A sharp increase in extinction was found in the region of a sucrose concentration between 30 and 40% and the maximum was at 34 and 35%. From this region, the highest content of specific virus protein and the highest purity (determined as the ratio of virus protein to total protein) were also measured. Overall, more than 50% of the specific antigen content determined in the starting material was recovered in these peak fractions.

Chromatographic purification:

The inactivated virus preparations (see above) were applied to a CS column that had been equilibrated beforehand with five column volumes of 50 mM phosphate buffer, pH 7.5. It was then washed with 10 column volumes phosphate buffer in order to eliminate unbonded material. Bound material was then eluted with the same phosphate buffer with stagewise admixing of increasing amounts of the same buffer with addition of 3M NaCl. Between 3.2 and 3.9% of the specific antigen and 79 to 83% of the total protein was recovered analytically in the flow during application of the virus material. In the wash buffer, 6-11% of the total protein and 0-2.3% of the antigen were found. More than 95% of the antigen is therefore bound to the column material. During elution with 0.6 to 1.8M NaCl, about 60.0% of the antigen was recovered, the highest purity was achieved during elution with 1.2M NaCl. Higher salt concentrations to 3M NaCl eluted additional, small amounts (<15%) of antigen with lower specific purity.

Purification by combination of chromatography and ultracentrifugation:

Combined eluate after 0.6 and 1.2M NaCl elution were subjected to ultracentrifugation for 2.5 h at 80,000 G from chromatographic purification as described above. The virus pellet was resuspended in 50 mM phosphate buffer pH 7.5 and analyzed. The total protein concentration of this preparation was reduced to 0.7% of the initial content and the degree of purity had been increased ten-fold by this step.

This virus preparation was subjected to gradient purification as described above. After fractionation a very similar gradient profile was found, as achieved after direct gradient purification. The tip of the virus peak, however, had shifted slightly and now was at 37% sucrose.

### Example 16

### Recovery of a Virus Isolate and Virus Multiplication of a Human Herpes Virus

By sterile puncture of a fresh herpes efflorescence in the blister stage (labial herpes blisters) with a tuberculin syringe, a minimal amount of tissue fluid was obtained and suspended according to Example 3 in standard medium with addition of antibiotics and filtered using a filter with a pore size of 0.45 μm. The filtrate was inoculated in a culture flask with 25 cm² culture surface with adherent MDCK 33016 cells in standard medium and incubated at 37° C. After 4 days samples of the supernatant were taken and after 7 days the entire supernatant of the cultures were taken and frozen at less than −70° C. A sample taken after 4 days was diluted 1:10 and then in steps of 10 in standard medium containing 10 μg/mL trypsin; 100 μL of these dilutions were introduced to the MDCK 33016 cells in standard medium. After 13 days of incubation at 37° C., a CPE was found in a few cultures of the first dilution step. The supernatant of these cultures were harvested and diluted again and inoculated in new cultures. After 6 to 9 days an increasingly more distinct CPE was found in several dilution steps of this third virus passage as typical herpes virus-plaques. A directly infected culture parallel with the same starting material with 175 cm² culture surface also showed exclusively the same typical plaques. For further cloning of the virus, this dilution process was repeated again, in which supernatant in cell cultures of the last positive dilution were used. In addition to harvesting of the culture supernatants, the remaining cells were fixed with a 3% formaldehyde solution for 16 h then incubated with 1% Triton X-100 for 30 min and then subjected to immunofluorescence investigations according to standard methods with specific, FITC-labeled monoclonal antibodies against HSV-1 (Biosoft product No. 17-088). It was found that only cells in the vicinity of the plaque had immunofluorescence. By this demonstration and by a specific PCR demonstration, the isolate was clearly identified as herpes simplex virus 1.

The cloned virus was further multiplied in standard medium in suspension cultures and used for production seed virus at a sufficient virus titer (>10⁶ infectious units/mL) as described in Example 3. The seed virus preparations regularly contained virus titers between 10⁷ and 10⁸ CID₅₀/mL. Determination of the virus titer occurred according to standard methods known to one skilled in the art in HEP-2 or Vero cells, but can also occur in adherent MDCK cells in which evaluation of the titrations is carried out with reference to typical plaques. The seed virus preparations were aliquoted at −70° C. or frozen below that and used for infection of production cells. The possibility of using the same MDCK cells and the same culture conditions in terms of media and additives as for later production is a significant advantage, since the documentation demands during registration of the corresponding products are significantly reduced and acceptance of the seed virus is improved.

### Example 17

### Production of Human Herpes Viruses

For infection of the production cells according to Examples 8 to 13 with herpes simplex virus 1 (isolate as described in the preceding example), a MOI of 0.1 or 0.01 and an incubation time of 48 to 96 h after harvest are chosen. However, lower or higher MOIs with correspondingly longer or shorter incubation times can also be used, in which the yields could vary somewhat since the optimal harvesting time is not always found. As a rule, however, the aforementioned conditions are preferred so that culture yields for economic reasons and for facilitation of subsequent workup do not lie significantly below 10⁸ 50% culture-infectious units/mL (CID₅₀/mL). Beyond this, this time scheme can be favorably adapted in normal work rhythms. Unduly low MOIs below 0.0001 and lengthened incubation times almost always lead to lower yields and are therefore suboptimal.

### Example 18

### Multiplication of Flaviviruses

Suspension cultures of MDCK 33016 cells with a cell density of 1-1.5×10⁶ cells/mL were infected under standard conditions (standard medium, 37° C. culture and infection temperature) with a Central European encelphalitis virus (strain K23, Niedrig et al., 1994, Acta Virologica 38:141-149). Deviating from the previous examples, strongly varying MOIs were used for infection. Moreover, the infection cultures were partly kept in chemically-defined medium or in medium without protein-containing additives. Different culture and harvesting methods were used which show that, even when different parameters are changed, high yields can be achieved with the system and even multiple harvests are possible. These changes are summarized in Table 3. Virus titration occurred in A 549 cells (ECACC No. 86012804) and was evaluated after 5 days with reference to CPE. The fact that the repeated harvest of the same culture was accompanied by exchange of the culture medium so that the cells during each harvest were supplied with new medium and could therefore grow further is worth noting. Without these harvests, the culture would not remain viable and productive over a longer period. Since frequent medium exchanges at short intervals could not compensate for the high metabolic output of the cultures, additional medium supplements and increases of the cultures occurred after 4 or 5 days of infection time.

TABLE 3
Multiplication of CEE virus/K23 in MDCK 33016 cultures in standard medium
and in alternative media using various MOI and harvesting variants.

| | Yield (log 10 CID₅₀/mL) during harvest after days | | | | | | | | |
|---|---|---|---|---|---|---|---|---|---|
| | Medium | | | | | | | | |
| MOI | 1 | 2 | 3 | 4 | 5 | 6 | 7 | 8 | Employed medium |

Lots with multiple harvests during complete media exchange

| | | | | | | | | |
|---|---|---|---|---|---|---|---|---|
| 2.0 | 9.0 | | 8.8 | | | 8.8 | | Standard medium |
| 2.0 | | 9.0 | | (M + 30)⁺ | | 8.4 | | Standard medium |
| 2.0 | | 6.1 | | (M + 30) | | 6.1 | | Protein-free medium |
| 0.2 | | 7.8 | | (M + 30) | | 7.8 | | Chemically-defined medium |
| 0.2 | 8.7 | | 8.0 | | | 7.7 | | Standard medium |
| 0.2 | 8.3 | | (M + 30) | 8.6 | | | | Standard medium |
| 0.2 | | 9.0 | | (M + 30) | 9.0 | | | Standard medium |
| 0.2 | 8.6 | | 9.2 | | | 9.0 | | Standard medium |
| 0.2 | 9.0 | | | 9.0 | | | 8.6 | Standard medium |
| 0.2 | | 7.3 | | (M + 30) | 8.2 | | | Protein-free medium |
| 0.2 | | 7.2 | | (M + 30) | 8.6 | | | Chemically-defined medium |

Lots with sampling without media exchange or supplementation

| | | | | | | |
|---|---|---|---|---|---|---|
| 10^{-0.3} | 7.7 | 8.3 | 9.2 | 9.4 | 9.3 | Standard medium |
| (=−0.5) | | | | | | |
| 10^{-0.3} | 6.3 | 7.5 | 8.4 | 8.6 | 8.9 | MEM medium, adherent |
| | | | | | | culture, 1% FCS |
| 10^{-1.3} | 5.2 | 6.3 | 6.6 | 6.8 | 6.8 | Standard medium, |
| (=−0.05) | | | | | | temperature exceeded due to |
| | | | | | | agitator |
| 10^{-1.3} | 5.1 | 6.2 | 7.1 | 8.0 | 8.4 | Standard medium |
| 10^{-2.3} | 4.8 | 6.2 | 7.6 | 7.5 | 8.1 | Standard medium |
| 10^{-3.3} | 3.4 | 4.7 | 4.9 | 5.6 | 6.0 | Standard medium |
| 10^{-4.3} | 2.7 | 3.7 | 4.3 | 4.3 | 4.4 | Standard medium |
| 10^{-5.3} | 2.5 | 2.6 | 3.4 | 3.7 | 4.3 | Standard medium |

⁺(M + 30) means medium supplementation + 30% of the culture volume on the stated day

### Example 19

### Multiplication of Picornaviruses

Adherent MDCK 33016 cultures were cultured for infection with hepatitis A virus (HAV, strain HM 175, ATCC VR-1358) in MEM medium with addition of 5% fetal calf serum and bicarbonate (cf. Example 2). In the context of the experiment, an additional “Munich” virus isolate was used (cf. Frosner et al., 1979, Infection 7:303-305). The diluted virus was inoculated into the freshly prepared culture and the culture incubated at 37° C. The cultures were subjected to further passage of 1:4 in alternating rotations of 3 to 4 days.

Suspension cultures of MDCK 33016 cells were cultured in standard medium according to Example 1, inoculated with HM 175 and incubated at 33° C. and then subjected to 1:10 passage weekly. The adherent cells in suspension cultures were further maintained after infection for up to 35 days. Detection of the active virus replication then occurred by means of CPE (strain HM 175) or according to an already described method (see Virus titration, page 93 in Gregersen et al., 1988; Med. Microbiol. Immunol. 177:91-100). A human anti-HAV antibody as purified IgG was used as virus-specific antibody as a deviation (designation F 86012, kindly finnished by Dade Behring). Product No. 39015 (Sigma Co.) was used as anti-human IgG antibody with biotin labeling. The specific detection of active virus multiplication with this system yields brownish-pink colored cells that are easy to recognize on low magnification in a microscope. Virus-negative cells on the other hand appear uncolored or have only a slight coloration. Virus titrations at 3 weeks after preparation were also evaluated with the same detection methods, for which human diploid cells (MRC-5) were used as the culture system.

In all the infection lots described above and with both virus isolates employed, an active HAV replication can be detected in the MDCK cells. A surprisingly rapid virus multiplication was detected with strain HM 175 in suspension cultures. On day 7 after infection, the measured virus titer in the supernatant was 10 4 CID₅₀/mL; this culture was subjected to 1:10 passage weekly by simple dilution and again yielded similar virus titers in the resulting cultures after 7 days. At the end of culturing and after two additional cell passages, the virus titer in one sample of the cell-free medium was determined. A sample of the entire culture was also taken and the cells contained in it broken down by two-fold freezing at −20° C. and thawing. The cell components were removed by centrifugation before the samples were titrated. The virus yields obtained from this lot are summarized in Table 4 and show that, without an adverse effect on specific yields, a weekly ten-fold multiplication of the cultures is possible, in which good virus titers per volume unit can be harvested despite the massive amount increase. A significant fraction of virus is then found in the supernatant, which is also surprising for this strongly cell-bound virus (see Table 4).

TABLE 4
Multiplication of hepatitis A virus (strain HM 175) in
MDCK 33016 suspension cultures with continuous
multiplication and increase in the culture volume.

| | Cell passage | Relative harvest | Total virus yield (CID₅₀) | |
|---|---|---|---|---|
| Day after | (increase in | volume | | After cell |
| infection | culture volume) | (day 0 = 1) | In medium | breakdown |
| 7 | 1:10 | 1 | 10^{7.4} | 10^{7.8} |
| 14 | 1:10 | 10 | 10^{8.5} | 10^{9.2} |
| 21 | 1:10 | 100 | n.d. | n.d. |
| 28 | 1:10 | 1000 | 10^{10.8} | 10^{11.4} |
| 35 | End | 10,000 | 10^{12.5} | 10^{14.2} |

n.d.: not determined

### Example 20

### Multiplication of Rhabdoviruses

Suspension cultures in standard medium according to Example 1 were seeded in cell culture flasks with a cell density of 1×10⁶ cells per mL of medium. After growing the cultures, two cultures were infected with a rabies virus (strain Pitman-Moore, vaccine virus strain) with a MOI of 0.01 and one culture of MOI of 0.001. The cultures were incubated at 37° C. and detached every 4 or 3 days with trypsin and subjected to passages in a 1:10 ratio (after 4 days) or 1:8 ratio (after 3 days) and maintained this way for 18 days (see Table 5). The infection success was followed at each passage. A culture was provided with 3.5% formalin solution and incubated for 3 days at room temperature in the solution in order to achieve inactivation of the viruses. After elimination of the formalin solution, the culture was washed with PBS and incubated for 25 min with 1% Triton X100 in PBS at room temperature. After removal of the solution, it was washed three times with PBS and an FITC-labeled antibody against rabies virus was applied (50 μL 1:400 diluted rabbit antirabies IgG FITC, Dade Behring, OSHY 005). After 90 min of incubation at 37° C., it was washed again with PBS and the culture evaluated under an inverted fluorescence microscope.

As an alternative, virus titrations of the culture supernatants were conducted according to standard methods in MRC-5 cells, which were also evaluated by immunofluorescence as described above after formalin/Triton pretreatment. By means of the virus titers achieved with this system, a rough correlation to the yield in the corresponding production methods was made using MRC-5 cultures for an approved human vaccine (Rabivac) which permits an orientation as to how much vaccine antigen is contained per mL of culture harvest (see Table 5).

After only 4 days both lots (MOI 0.01 and 0.001) showed positive results and then a similar infectious course, but at the lower MOI the infectious courses—recognizable in the virus titers that were lower up to day 11 at about 1.2 to 0.5 log CID₅₀—were slightly slowed. From the third passage of the cultures on day 11, a very intense specific immunofluorescence with incipient cell destruction was found in all cultures, which then further increased until most of the cells had been fully destroyed by the fifth passage on day 18 so that the infection was terminated. The content of specific virus continuously rose to day 14 to then diminish again as a result of increasing cell destruction. The results of this infectious course are summarized in the following table and show that (measured on the known slow virus multiplication of rabies viruses) a very rapid virus multiplication without adaptation is be expected in these cells, in which good antigen yields can be harvested despite continuing remultiplication of the cells at regular intervals and repeatedly.

TABLE 5
Multiplication of rabies virus in MDCK 33016 cultures
during continuous enlargement of the culture volume.

| Day | Passage | Relative | Rabies antigen |
|---|---|---|---|
| after infection | of the cells | culture volume | (vaccine doses/mL) |
| 4 | 1:4 | 1 | not determined |
| 7 | 1:3 | 4 | not determined |
| 11 | 1:4 | 12 | 0.2-0.4 |
| 14 | 1:3 | 36 | 0.4-0.5 |
| 18 | not applicable | 108 | 0.4-0.5 |

In similar fashion the same virus was directly inoculated in suspension cultures according to Example 1 in which a MOI of 0.0001 was additionally used. Standard medium was exclusively used again for the entire infectious course and the cultures were also transferred twice weekly at 1:8 or 1:10. Transfer occurred only by simple dilution of the cells in fresh medium and seeding anew. The infection success was followed here only with reference to virus titrations in MRC-5 cells as described above. The infections at all three MOIs after only 4 days yielded positive virus titers in the culture supernatant. The virus titers rose after initial dilution loss after the seventh day from passage to passage and despite the again conducted exponential dilution continuously rose but led to no massive cell destruction in the suspension cultures. The infection was followed to the eighth passage (day 28 after infection) and then interrupted.

Virus samples from these infections were frozen as seed virus and used for a new infection of suspension cultures beginning with 100 mL and also in the standard medium and under the same passage conditions as described above. The MOI was reduced in this case to 0.000025. The infection was maintained over six cell passages (21 days). Virus titers which, converted, gave about 0.3 vaccine doses per mL of culture supernatant were measured at the end of this infectious course with slowly rising virus titers despite the massive passage dilutions. If the entire culture volume and not just a part of it had been subjected to further passages, about 500 L of culture could have been harvested after six passages, which would have been a virus yield corresponding to about 150,000 vaccine doses.

### Example 21

### Multiplication of Paramyxoviruses

As representative of the paramyxoviruses, the ATCC VR-288 strain was used. The third day was selected as the harvest time, since this virus replicates very rapidly. The MDCK 33016 cells also proved to be a very suitable titration system for the paramyxovirus with more efficient virus replication in MEM medium without serum or protein addition, but with bicarbonate addition.

Evaluation and titration occurred after 5 days. Cultures that were further incubated after infection at 37° C. gave yields of 10^{7.4} CID₅₀/mL; the same titers were measured if the infection temperature was reduced to 33° C. from the infection time point.

With this virus a direct comparison between adherent and suspension cultures was carried out. The maximum yield in the adherent system was 10^{6.6} CID₅₀/mL after 96 h of infection time, the suspension culture system gave in comparison much better and more rapid yields of 10^{7.3} CID₅₀/mL after 72 h.

As an alternative, adherent MDCK 33016 cells according to Example 2 were infected with MEM with 5% FCS with another virus of the same family (PI-3, ATCC VR-93). After 1 week of incubation at 37° C., the supernatants contained at least 10⁶ CID₅₀/mL after titration in CV-1 cells (ECACC 87032605), showed a positive hemagglutination with guinea pig erythrocytes and a positive immunofluorescence with specific antibodies (anti-PI-3 MAb-FITC from the Biosoft Co.).

The same virus strain (PI-3, ATCC VR-93) was also used under chemically-defined and protein-free media in similar fashion to Example 12 for infection in MDCK 33016 cultures. On the infection days 3, 5, 9 and 12, 22% of the culture volume was removed and replaced by fresh medium. On day 7, 50% of the culture volume including the cells was removed and replaced with new medium. Overall the culture volume during infection was completely exchanged more than once and offered the opportunity by medium supplementation to further multiply the cells according to the dilution. The method employed corresponds overall to a roughly 1:2.4 passage of the culture in which only the excess amounts were removed. The significantly higher passage or dilution of the culture, possible especially in the initial phase, was clearly not fully exploited here.

The following virus yields were measured.

Day of infection:

| | 3 | 5 | 7 | 9 | 12 | 14 |
|---|---|---|---|---|---|---|
| log CID₅₀/mL: | 7.9 | 8.05 | 8.25 | 7.45 | 6.7 | 7.0 |

(average values from duplicate tests)

### Example 22

### Multiplication of Reoviruses

Suspension cultures of MDCK 33016 cells in standard medium were infected with reovirus type 3 (obtained from Bio Doc, Hannover) at a MOI of 0.01 and further incubated for 3 or 5 days at 33 or 37° C. Samples of the culture supernatants were taken after 5 and 7 days and titrated in the system furnished using BHK cells in MEM medium with 3% FCS. Evaluation of the titrations occurred after 7 days.

The virus yields of the suspension cultures after 5 days at 37° C. were 10^{8.1} CID₅₀/mL, at 33° C. 10^{8.0} CID₅₀/mL. After 7 days the titers in both temperature lots were at 10^{8.0} CID₅₀/mL.

The same virus strain was used under chemically-defined and protein-free media similar to Example 12 in MDCK 33016 cultures for infection at a MOI of 0.01. On the infection days 3, 7 and 10, 22% of the culture volume was removed and replaced by fresh medium. On day 7, 50% of the culture volume including the cells was removed and replaced with new medium. Overall the culture volume during infection was therefore almost completely exchanged and offered the cells an opportunity by medium supplementation to further multiply according to the dilution. The method employed corresponds to a roughly 1:2 passage of the culture in which only the excess amounts were removed. The significantly higher passage or dilution of the culture, possible especially in the initial phase, was clearly not fully exploited here.

The following virus yields were measured.

Day of infection:

| | 3 | 7 | 10 | 14 |
|---|---|---|---|---|
| log CID₅₀/mL: | 5.4 | 7.1 | 6.6 | 6.6 |

(average values from duplicate tests)

### Example 23

### Multiplication of Pneumoviruses

Adherent MDCK 33016 cultures in MEM medium with addition of 5% FCS and bicarbonate (cf. Example 2) were used for infection with human RSV-A (strain A-2; ATCC VR-1302). The virus was diluted 1:100 and inoculated into the freshly prepared culture and the culture then incubated at 37° C. After a week 1 mL of the culture supernatant was transferred to a new culture and again incubated for 7 days. The harvested culture supernatant in MA-104 cells (ECACC 85102918) shows during evaluation of the titration a virus titer of 10^{5.5} CID₅₀/mL by means of CPE.

The virus strain A-2, ATCC VR-1302 was used for infection under chemically-defined and protein-free media similar to Example 12 in MDCK-33016 cultures. On infection days 3, 5, 7, 9 and 12, 22% of the culture volume was taken and replaced by fresh medium. On day 7, 50% of the culture volume including the cells was removed and replaced by new medium. In all, the culture volume during infection was exchanged completely more than once and gave the cells an opportunity by medium supplementation to further multiply according to the dilution. The method employed corresponds overall to a roughly 1:2.4 passage of the culture in which only the excess amounts were removed. The significantly higher passage or dilution of the cultures possible, especially in the initial phase, was clearly not fully exploited here.

The following virus yields were measured:

Day of infection:

| | 3 | 5 | 7 | 9 | 12 | 14 |
|---|---|---|---|---|---|---|
| log CID₅₀/mL: | 7.85 | 8.5 | 7.55 | 6.55 | 4.45 | n.t. |

(average values from duplicate tests)
n.t.: Samples not tested, some unsterile

The virus strain RSV-B, ATCC VR-1401 was tested in an equivalent lot. For virus titration Hep-2 cells (subline Hep-2C, kindly furnished by the Paul Ehrlich Institute, formerly Frankfurt) was used, since the typical viral syncytia are better developed in it and evaluation is therefore facilitated.

The following virus yields were measured:

Day of infection:

| | 3 | 5 | 7 | 9 | 12 | 14 |
|---|---|---|---|---|---|---|
| log CID₅₀/mL: | 3.7 | 4.75 | 7.45 | 6.3 | 3.2 | 3.75 |

(average values from duplicate tests)

1. Method for multiplication of viruses in cell culture, comprising steps in which
(a) cells are infected with a virus;
(b) after infection the cells are cultured in cell culture under conditions that permit multiplication of the viruses and at the same time targeted additional, at least two-fold, multiplication of the cells.

2. Method according to claim 1, characterized in that after infection the cells are cultured under conditions that cause at least five-fold multiplication of the cells.

3. Method according to claim 2, characterized in that after infection the cells are cultured at least 7 days in cell culture.

4. Method according to claim 1, characterized in that during multiplication of the viruses and multiplication of the cells, fresh medium, medium concentrate or media components are added at least once or at least part of the viruses and cells are transferred to a culture vessel that contains fresh medium, medium concentrate or media components.

5. Method according to claim 4, characterized in that the addition of the medium, medium concentrate or media components or transfer of the cells and viruses to another culture vessel is repeated at least once.

6. Method according to claim 4, characterized in that addition of the medium, medium concentrate or media components or transfer of the cells and viruses to another culture vessel is repeated several times.

7. Method according to claim 4, characterized in that medium is removed on multiplication of the viruses and cells.

8. Method according to claim 1, characterized in that during multiplication of the viruses and cells, medium is continuously removed and fresh medium, medium concentrate or media components are added.

9. Method according to claim 1, characterized in that the cells are MDCK cells.

10. Method according to claim 1, characterized in that before infection the cells are cultured adherently or as a suspension culture.

11. Method according to claim 1, characterized in that after infection the cells are cultured adherently or as a suspension culture.

12. Method according to claim 9, characterized in that the MDCK cells originate from the cell line MDCK 33016.

13. Method according to claim 1, characterized in that the virus is a ssDNA, dsDNA, RNA(+), RNA(−) or dsRNA virus.

14. Method according to claim 1, characterized in that the virus is chosen from adenoviruses, ortho- or paramyxoviruses, reoviruses, picornaviruses, enteroviruses, flaviviruses, arenaviruses, herpes viruses or pox viruses.

15. Method according to claim 14, in which the cells are infected with an adenovirus, polio virus, hepatitis A virus, Japanese encephalitis virus, Central European encelphalitis viruses and the related eastern (Russian or other) forms, dengue virus, yellow fever virus, hepatitis C virus, rubella virus, mumps virus, measles virus, respiratory syncytial virus, vaccinia virus, influenza virus, rotavirus, rhabdovirus, pneumovirus, reovirus, herpes simplex virus 1 or 2, cytomegalovirus, varicella zoster virus, canine adenovirus, Epstein-Barr virus, bovine or porcine herpes viruses, BHV-1 virus, pseudorabies virus, or rabies virus.

16. Method according to claim 1, characterized in that the virus has a viral genome comprising a sequence that codes for a heterologous functional protein with a size of at least 10 kd.

17. Method according to claim 4, characterized in that the medium is serum-free.

18. Method according to claim 4, characterized in that the medium is a chemically defined medium.

19. Method according to claim 4, characterized in that the medium is protein-free.

20. Method according to claim 1, characterized in that multiplication of the viruses is carried in a perfusion system.

21. Method according to claim 1, characterized in that multiplication of the viruses is carried in a batch system.

22. Method according to claim 9, characterized in that the MDCK cells are cultured at temperatures between 30 and 40° C. for multiplication of the viruses.

23. Method according to claim 9, characterized in that the MDCK cells are cultured at an oxygen partial pressure between 35 and 60% for multiplication of the viruses.

24. Method according to claim 4, characterized in that the pH value of the medium lies between pH 6.8 and pH 7.8 for multiplication of the viruses.

25. Method according to claim 9, characterized in that the virus is introduced to the MDCK cells by infection with a MOI value between 10⁻⁸ and 10.

26. Method according to claim 1, characterized in that the viruses or a protein expressed by them are purified from the culture supernatant or the harvested cells.

27. Method according to claim 26, characterized in that the cells are MDCK cells and at least part of the culture medium is separated from at least part of the MDCK cells for purification of the viruses or protein.

28. Method according to claim 27, characterized in that the separation occurs by means of a deep bed filter or a separator.

29. Method according to claim 26, characterized in that the purification includes ultracentrifugation for concentration of the viruses.

30. Method according to claim 26, characterized in that the purification includes chromatography.

31. Method according to claim 26, characterized in that the viruses are inactive during purification.

32. Method for production of a drug or diagnostic agent, characterized in that it includes a method according to claim 1.

33. Method for production of a drug or diagnostic agent according to claim 32, characterized in that that the viruses or the protein are mixed with an appropriate adjuvant, auxiliary, buffer, diluent or drug carrier.

34. The method of claim 2, characterized in that after infection the cells are cultured under conditions that cause at least ten-fold multiplication of the cells.

35. The method of claim 3, characterized in that after infection the cells are cultured at least 21 days in cell culture.

36. The method of claim 35, characterized in that after infection the cells are cultured at least 28 days in cell culture.

37. The method of claim 36, characterized in that after infection the cells are cultured at least 35 days in cell culture.
